(19) United States
(12) Patent Application Publication
(10) Pub. No.: US 2003/0108487 A1
(43) Pub. Date: Jun. 12, 2003

(54) Device for spraying a cosmetic product

(76) Inventor: Isabelle Bara, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/203,781

(22) PCT Filed: Nov. 22, 2001
(86) PCT No.: PCT/FR01/03689

(30) Foreign Application Priority Data
Dec. 15, 2000 (FR) 00/16397

(51) Int. Cl. A61K 9/00; A61K 7/021; B65D 83/06; B65D 83/14

(52) U.S. Cl. 424/47; 424/63; 222/401

(57) ABSTRACT

A self-contained device of unitary structure for spraying a cosmetic substance onto a keratinous medium, in particular the skin, the device comprising a supply of substance to be sprayed and a receptacle containing a gas, together with a valve making it possible, when actuated, to spray the substance, the substance containing solid particles and at least one dispersing or jelling agent.

The invention relates to a self-contained portable device for spraying a cosmetic onto a keratinous medium, in particular the skin, the lips, or the fingernails.

### BACKGROUND OF THE INVENTION

The use of a vector gas for spraying paint has been known for a long time, in particular from U.S. Pat. No. 2,635,921. That technique has been thoroughly developed and constitutes the subject matter of numerous patent applications. As an indication, mention may be made of European patent application No. EP-A1-0 208 247 and U.S. Pat. Nos. 5,255,852 and 5,713,519.

U.S. Pat. No. 1,430,506 discloses a device which comprises a tank on which two nozzles are fixed that are connected to a compressed air inlet, the two nozzles being suitable for emitting jets of air that meet in a region situated over a substance feed orifice. Such a device requires an external source of compressed air and a feed hose.

It is well known to apply makeup by means of an air brush comprising a spray stylus connected to a cylinder of compressed vector gas, the spray stylus comprising firstly a nozzle for ejecting the vector gas and secondly a container or cup containing the substance to be sprayed, said substance being progressively sucked up through a duct in the cup by means of the suction created by the Venturi effect due to the speed of the vector gas at its outlet from the nozzle.

Airbrush type spraying may be used in particular make the boundary between zones that have been treated and zones that have not been treated less visible, to achieve blending, shading, or impressions of volume.

The hand actions involved are also different since the substance which is sprayed does not need to be spread out once it has been deposited on the keratinous medium.

The substance may thus be supplied in very hygienic manner since there is no need for contact with the fingers or with an applicator.

A device of the kind described above constitutes the subject matter of Canadian patent application CA-A-2 152 406, for example.

Similar devices are sold by DINAIR under the registered trademark BEAUTY ART and BODY ART, with the cylinder of compressed vector gas being replaced by an air compressor.

Those known devices in which the spray stylus and the force of vector gas do not constitute a self-contained structure given that they are interconnected by a hose. They are relatively bulky, and they are intended mainly for professional use. They are unsuitable for being carried about easily by a woman in her handbag, like conventional makeup accessories.

French patent application FR-A-2 781 208 discloses a self-contained portable device for spraying makeup in which the vector gas is present in liquefied form in a receptacle, with the substance to be sprayed and the vector gas being stored separately.

When making use of a vector gas that is stored in the liquefied state, the length of time the device may be used is extended and the cooling that accompanies expansion of the vector gas contributes to producing a feeling of freshness at the time of application.

The substance may be brought into contact with the vector gas by means of a duct or by capillarity by means of a felt.

In such a self-contained portable device, spraying the substance gives rise to numerous difficulties.

Firstly, the rate at which the substance is brought into contact with the vector gas in order to be entrained thereby must not be too great since otherwise the spray runs the risk of forming droplets that are too large in size or that are irregular, nor must said rate be too small since vector gas consumption then increases rapidly and the length of time the device may continue to be used becomes insufficient.

A felt is often used to bring the substance into contact with the vector gas, since that enables the flow rate of the substance to comply fairly well with these contradictory requirements.

Nevertheless, it is not always desirable to use a felt, particularly if the substance contains a large fraction of solid particles since the felt is then liable to behave as a filter which retains the solid particles.

If the felt is replaced by a duct, either because the substance is too viscous or because it contains too large a fraction of solid particles, then there is a danger of the duct becoming clogged if its section is too small. However the section of the duct used must not be too large since that leads on the contrary to a risk of the spray having droplets that are too large or of irregular size, which spoils the quality of the makeup.

U.S. Pat. No. 4,742,963 describes another self-contained portable device in which the vector gas and the substance are stored in a common receptacle in contact with each other. It is recommended to shake the receptacle when necessary in order to homogenize the substance before using the device. In that known device, the substance to be sprayed is not sucked up by suction created by the Venturi effect, but is ejected under pressure into an ejection nozzle. The above-mentioned problems do not arise, but the substance is liable to clog the ejection nozzle, because of the absence of vector gas.

Japanese patent application JP-A-63287711 describes a cosmetic emulsion for spraying, the emulsion containing lecithin in order to make application more uniform. That application does not deal with the difficulties mentioned above that are encountered with self-contained portable devices such as the device described in application FR-A-2 781 208.

### OBJECTS AND SUMMARY OF THE INVENTION

Consequently, there exists a need to benefit from a self-contained portable device capable of spraying a substance containing at least 0.3% by weight of solid particles, with good spray properties, and capable of obtaining a good makeup result, which device must have low risk of malfunctioning.

In particular, there exists specifically a need to benefit from a self-contained portable device in which the substance is sprayed by suction produced by the Venturi effect by a vector gas, which device must be capable of operating over lengths of time that are compatible with the expectations of users and with a substance that contains a relatively high fraction of solid particles, such as a foundation makeup, without any risk of the substance feed duct becoming clogged even in the event of the device being stored for a long period and in the absence of the substance being stirred or stirred only moderately at the time of use.

The novel self-contained device of unitary structure of the invention serves in particular to satisfy this need and it comprises a supply of substance to be sprayed and a receptacle containing a vector gas, together with a valve making it possible, when actuated, to spray the substance, the substance containing solid particles and at least one dispersing or jelling agent.

Because the substance contained in the self-contained portable device contains not only solid particles but also a dispersing or a jelling agent, it is possible to ensure that the substance is sufficiently fluid to be extractable, in particular by suction by the Venturi effect by means of a vector gas, while preventing the solid particles from agglomerating in such a manner as to be liable to clog the ducts through the substance for spraying is brought into the suction zone.

The presence of a dispersing or jelling agent makes it possible in particular to avoid or to slow down sedimentation or the appearance of a new phase or cream within the substance while it is in storage, and thus encourages uniform extraction of the substance throughout operation of the device.

The dispersing or jelling agent is preferably selected in such a manner as to ensure that it does not crystallize in the medium containing it at ambient temperature and under conditions of use.

Ambient temperature covers the range of temperatures that correspond to extreme conditions of use, typically the range 10° C. to 45° C.

Jelling or dispersing agents that might crystallize in the medium at ambient temperature and that are preferably not used to implement the invention include the following:

waxes, in particular hydrocarbon waxes or silicone waxes (alkyl or alkoxydimethicone);

fatty acid and fatty alcohol esters, including cerides and sterides or glycerophosphocholine and fatty acid esters, such as phospholipids; and

amino and fatty acids, in particular ceramides.

The substance may comprise more than 0.3% solid particles for example 5% to 70% by weight of solid particles, preferably 20% to 70% and more preferably still 30% to 60% when the substance is a foundation makeup, in particular, the content of the dispersing or jelling agent preferably lying in the range 0.01% to 10% by weight, and preferably being greater than or equal to 0.1%.

The particles contained in the substance may be colored or uncolored.

In a particular embodiment of the invention, the substance contains pigments, optionally a plurality of pigments of different colors.

When the substance contains pigments of different colors, the presence of at least one dispersing or jelling agent makes it possible to reduce separation of pigments of a given color from pigments of another color, thus making it possible to obtain makeup of tone that remains constant over time.

The particles may be pigments such as, for example: iron, titanium, or zinc oxide, organic varnishes or inorganic pearlescent agents, mica, titanium, or titanium-mica, for example.

The particles may also be organic powders, in particular particles of polyamide, of polyethylene, of polyacrylate, of methyl methacrylate, of polyurethane, of cross-linked polystyrene, or mixtures thereof, for example.

The particles may also be powders of mineral origin such as talc, clay, silica, boron nitride, or mixtures thereof, or powders of vegetable origin such as starch, or powders of animal origin such as seashell powder.

The jelling agents may be modified where appropriate by organic groups in order to jell oils.

The particles may also be silicones in the form of beads of methylsesquioxane resin as sold under the trade name “TOSPEARL” by Toshiba, or cross-linked silicones, e.g. in the form of an aqueous dispersion as described in U.S. Pat. No. 5,928,660, in particular.

The particles may also come from fluorine-containing powders such as powdered PTFE (polytetrafluoroethylene).

The particles may be of nanometer size, e.g. comprising nanotitanium or nanoiron oxides, or they may be of micrometer size. The substance may comprise particles both of micrometer size and of nanometer size.

The particles may optionally be coated.

As mentioned above, the substance preferably comprises 0.01% to 10% by weight of jelling and/or dispersing agent(s) as a function of the nature of the other ingredients in the substance, so as to ensure that the viscosity of the substance is compatible with the way in which the substance is extracted from the supply thereof, in particular with the substance being extracted by being sucked out by the Venturi effect.

The dispersing and/or jelling agent(s) used may be hydrophilic or lipophilic.

As a jelling agent, it is possible to use in particular, hydrophilic jelling agents of polymeric nature such as, for example, jelling agents of the polysaccharide type, e.g. xanthan gum, gellan gum, guar gum or its derivatives, cellulose and its derivatives, jelling agents of the vinyl type, carboxyvinyl type, acrylic type, polyurethane type, mineral type jelling agents of non-polymeric nature, such as clays, e.g. Veegum® sold by Vanderbilt, optionally modified Bentone® sold by Rheox, or hydrophilic silicas.

It is possible in particular to use as lipophilic jelling agents, agents of the polymer type or of the “organo-jelling” type, i.e. in the form of non-polymeric small-sized molecules that establish interactions in such a manner as to build up lattices of the kind mentioned in the book “Specialist surfactants” by Blackie Academic and Professional, Chapter 8.

As a jelling agent of the organo-jelling type, mention may be made of 12 hydroxystearic acid, its salts and derivatives (esters or amides), amino acid amides or N-acid amino acids, amides of tricarboxylic acids, sorbitol dibenzylidene, or alditol and derivatives thereof.

As examples of lipophilic jelling polymers, mention may be made of silicone polymers an derivatives thereof (polydimethylsiloxane (PDMS) of high molecular weight, greater than 10,000), said silicone polymers optionally being linear (“gum”) as contained in the product referenced Q2 1401 from Dow Corning or cross-linked, of the “resin” type, such as those contained in the product referenced KSG 6 or 16 from Shin Etsu or those known under the reference TREFIL 505 C or 506 C from Dow Corning, or indeed those known under the reference SR 5 CYC from Gransil.

It is also possible to use polymer type lipophilic jelling agents such as polymers derived from silicones or acrylic silicones.

It is also possible to use lipophilic jelling polymers such as polycondensates of polyamide type or polysaccharides having hydrophobic chains or polyurethanes.

When one or more dispersing agents are used, they should be of hydrophilic or lipophilic type and act by electrostatic repulsion or by steric stabilization.

As hydrophilic dispersing agents, mention may be made in particular of those having at least one anionic filler, e.g. carboxylic dispersants, sulfonates, terpolymers of acrylic acid, polyaspartates, derivatives of maleic acid, these dispersing agents acting mainly by electrostatic repulsion.

Among dispersions that act more by steric stabilization, mention may be made of polyvinyl pyrrolidone (PVP) type dispersants, polyoxyethylene, and polycaprolactone.

Dispersants used in oily media could be carboxylates, acrylics, hydrocarbons including a ring, e.g. styrene, or of the succinimide type.

The jelling or dispersing agents mentioned above may be used separately or in mixtures.

The substance may include at least one dispersing agent and at least one jelling agent.

In general, the supply of substance is advantageously contained in a receptacle that is sufficiently closed to enable the substance to be conserved in the device, while not in use, for a length of time in excess of at least 1 month at ambient temperature.

Advantageously, the supply of substance is contained in a removable cartridge.

In a variant, the supply of substance may be contained in a non-removable receptacle.

The viscosity of the substance preferably lies in the range 20 centipoises to 500 centipoises, i.e. in the range 20 millipascals per second (mPa/s) to 500 mPa/s), with viscosity being measured using an RM 180 rheometer from Rheometric Scientific, of the rotary type, using a suitable moving unit of “1”, “2”, or “3” type depending on the fluidity of the formulation, after 10 minutes at a shear rate of 200/s, with measurements being performed at a temperature of 25° C.

The substance may further comprise any of the usual compounds conventionally used in cosmetics, in particular hydrophilic or lipophilic cosmetic active agents such as, for example, UV blocking agents (filters), or moisturizers (e.g. glycerins), care oils, antioxidants, preservatives, perfumes, an anti-foaming agents, this list not being exhaustive.

The device may include a dip tube dipping into the supply of substance.

The substance may be contained in the flexible bag placed inside a receptacle containing the gas.

In a variant, the supply of substance may be contained in a receptacle which is different from the receptacle containing the gas.

The receptacle may be fitted with a valve having at least three positions, namely: a rest position; a position enabling the substance to be dispensed together with the vector or propellant gas; and a position enabling vector gas to be dispensed on its own.

The device may have at least one substance feed duct and at least one vector gas feed duct, preferably two vector gas feed ducts, the ducts being arranged in such a manner that the vector gas leaving the vector gas feed duct generates a pressure drop suitable for sucking up the substance coming from the substance feed duct.

The device may comprise a supply of vector gas, at least one substance feed suitable for being put into fluid communication with a supply of the substance, the substance being sucked from the supply by suction established in the vicinity of said at least one substance feed by emission of the vector gas.

The device may further comprise at least one shutter suitable for interrupting fluid communication between said at least one substance feed and the supply of substance.

The conditions under which the substance is conserved in the supply of substance may thus be improved, and in particular it is possible to prevent volatile components from escaping.

It is also possible to avoid the risks of substance leaking out while the device is being carried about in a horizontal or an upside-down position, as may happen for example in a handbag.

The substance feed may comprise at least one orifice.

The device may be arranged in such a manner as to reestablish fluid communication automatically between said at least one substance feed and the substance supply whenever the vector gas is emitted. The device may thus be arranged in such a manner that fluid communication is automatically interrupted whenever vector gas emission stops.

The device may include a pushbutton suitable for acting simultaneously, whether directly or indirectly, on a vector gas dispenser valve and on the shutter so that fluid communication between the substance feed and the supply of substance is established when the user presses on the pushbutton to deliver the vector gas.

The supply of substance may be contained, for example, in a first receptacle fixed to a second receptacle containing the vector gas. The two receptacles may also have at least one portion in common, e.g. a partition defining at least two compartments respectively containing the vector gas and the substance to be sprayed.

The receptacle containing the substance and the receptacle containing the vector gas may be in fixed relationship, neither being capable of moving relative to the other in use. By way of example this makes it possible to obtain an assembly that is relatively compact and easy to carry about, in particular in a handbag.

The receptacle containing the substance may, for example, be annular in shape so as to leave a passage, e.g. a central passage, along which a control member for a valve fitted to the receptacle containing the vector gas may extend.

The shutter may be operationally connected to at least one element actuated by moving the pushbutton of the device. Such an element may comprise a hollow rod, for example, the rod having at least one internal passage enabling the substance contained in the supply of substance to reach the substance feed.

By way of example, the shutter may comprise at least one gasket suitable for closing at least one orifice through which the substance may flow to reach said at least one substance feed, and suitable for releasing said orifice at least in part while the substance is being dispensed.

In a particular embodiment, the shutter is constituted by a gasket mounted on a hollow rod, the rod being closed at its bottom end, the gasket being capable of bearing via its top face against a shoulder of the rod. The rod is pierced by at least one substance admission orifice, for example, of a diameter that is smaller than or equal to the thickness of the gasket. The gasket is suitable for coming into abutment via its bottom face against a fixed bearing wall when the hollow rod is pushed down, so that the gasket is then compressed and disengages the admission orifice at least in part, so as to allow substance to flow towards said at least one substance feed. By way of example, the gasket may also press via its periphery against the inside surface of a duct in which the hollow rod may move axially, said duct being capable of communicating freely with the outside, in which case the gasket makes it possible to obtain leaktight closure of the gap situated between the hollow rod and the inside surface of the duct. The top end of the duct may connect to a cover closing the top of the substance containing receptacle, for example. The hollow rod may be actuated by moving the pushbutton that controls vector gas emission, for example. The inside space at the bearing wall against which the gasket may come into abutment via its bottom face may communicate with the receptacle containing the substance, e.g. via an annular channel formed in a bottom end wall of the receptacle. The above-mentioned duct may be connected in leaktight manner to the above-mentioned tubular bearing wall.

According to an aspect of the invention, the device may comprise a pushbutton made by assembling together a bottom portion and a top portion. The bottom portion may be made integrally with an actuator rod, for example, and may extend in a passage of the receptacle containing the substance, e.g. a central passage. The control rod for the valve delivering the vector gas may be engaged in the actuator rod of the pushbutton.

The supply of substance may initially contain 5 cubic centimeters (cm³) to 200 cm³ of substance for example, or indeed 10 cm³ to 100 cm³.

The gas may present at least one liquid phase inside the receptacle containing the vector gas.

### BRIEF DESCRIPTION OF THE DRAWINGS

Other characteristics and advantages of the present invention will appear on reading non-limiting descriptions of embodiments of the invention, and on examining the accompanying drawings, in which:

FIG. 1 is a diagrammatic perspective view of a spray device constituting a first embodiment of the invention;

FIG. 2 is a fragmentary diagrammatic plan view of the spray head of FIG. 1;

FIGS. 3 and 4 are fragmentary sections showing two variant embodiments for the spray head;

FIG. 5 is a diagrammatic view partially in section of a spray device constituting a second embodiment of the invention;

FIG. 6 is a diagrammatic view partially in axial section showing a spray device comprising a third embodiment of the invention;

FIG. 7 is a plan view of the FIG. 6 device;

FIG. 8 is a view analogous to FIG. 6, showing the device while spraying substance;

FIG. 9 is a diagrammatic axial section view showing a device constituting a fourth embodiment of the invention;

FIGS. 10 and 11 are diagrammatic axial sections showing a three-position valve;

FIG. 12 is a fragmentary diagrammatic axial section on XII-XII of FIG. 13, showing another embodiment of a spray device;

FIG. 13 is a diagrammatic and fragmentary plan view as seen looking along arrow XIII in FIG. 12;

FIG. 14 is a fragmentary and diagrammatic axial section on XIV-XIV of FIG. 13;

FIG. 15 is a fragmentary diagrammatic exploded perspective view of the FIG. 12 device;

FIG. 16 is a view on a larger scale showing a detail XVI of FIG. 12;

FIGS. 17 and 18 are diagrams showing other examples of shutters;

FIG. 19 is a diagram of an example of a microleak; and

FIG. 20 is a diagram of an example of an air intake valve.

### MORE DETAILED DESCRIPTION

In the following description, including the claims, the expression “comprising a” must be understood as meaning “comprising at least one”.

FIG. 1 shows a spray device 1 constituting a first embodiment of the invention, comprising a first receptacle 2 containing a supply of vector gas and a second receptacle 3 containing a liquid to be sprayed.

In this example, the vector gas is present in the liquefied state in the receptacle 2 and its nature is selected in such a manner as to be compatible with spraying onto a keratinous medium such as the skin, the fingernails, or the hair.

By way of example, the vector gas is compressed gas, in particular air, butane, isobutane, isopropane, or a fluorine-containing compound.

The vector gas can be present in the liquefied state.

Naturally, it would not go beyond the ambit of the present invention for some other vector gas to be used, for example compressed nitrogen.

In accordance with the invention, the substance contained in the receptacle contains at least 0.3% by weight of solid particles and at least one dispersing or jelling agent.

Reference can usefully be made to the beginning of the description where numerous examples of compounds are given that are suitable for being present in the substance.

In addition to the receptacles 2 and 3, the spray device 1 comprises a dispenser assembly 4 comprising a spray head 5 and an actuator member 56 such as a pushbutton, actuating a control valve that is not visible in the drawing.

The spray head 5 has a central nozzle 6 for dispensing the liquid contained in the receptacle 3 and two lateral nozzles 7 for emitting the vector gas.

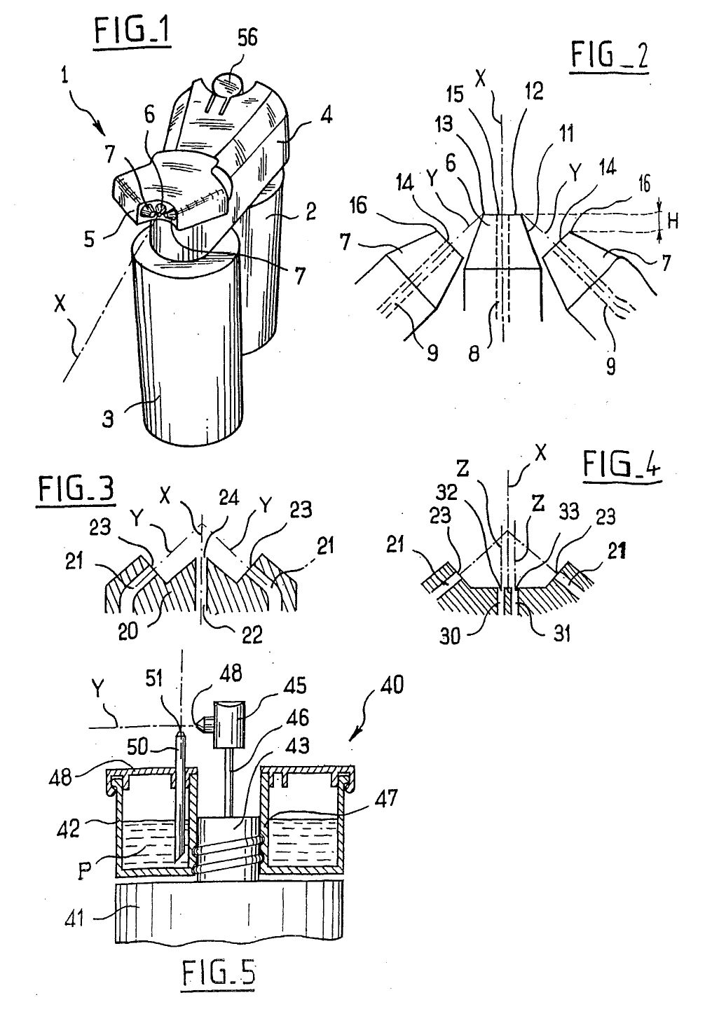

The nozzles 6 and 7 are shown in greater detail in FIG. 2.

The nozzle 6 has an internal passage 8 on an axis X, which axis coincides with the spray axis.

Each of the nozzles 7 has an internal passage 9 on an axis Y, the axes Y each making an angle of 45° with the axis X, and being contained in the same plane as the axis X.

The passage 8 is permanently in communication with the substance contained in the receptacle 3, the passage being extended into the inside of the receptacle by a dip tube, and the passages 9 communicate with the vector gas contained in the receptacle 2 via the valve actuated by the pushbutton 56.

The outlet orifices 14 for the vector gas have a diameter of 0.4 mm, for example, and the outlet orifice 15 for the liquid substance has a diameter of 0.9 mm, for example.

When the user presses on the pushbutton 56, the vector gas flows along the passages 9 and expands on leaving the nozzles 7, thus producing a pressure drop by the Venturi effect in front of the nozzle 6, thereby sucking substance into the passage 8.

Because of the way the axes Y are oriented, the vector gas jet emitted by the nozzles 7 meet each other, thus making it possible to obtain a spray having droplet characteristics and overall spray shapes that are compatible with obtaining satisfactory makeup results.

In the particular configuration shown in FIG. 2, the nozzle 6 also acts as a deflector-forming element and deflects a fraction of each of the vector gas jets emitted by the nozzles 7 in a forward direction.

In other words, at least a part of each vector gas jet strikes the nozzle 6 and then takes up a direction that is closer to the direction of the spray axis.

It will be observed that the nozzle 6 presents a hollow frustoconical portion 11 whose generator line makes an angle with the spray axis X which is smaller than the angle made by each of the axes Y with the axis X.

In the example shown in FIG. 2, the nozzle 6 has an end face 12 which is plane and perpendicular to the axis X, and the axis Y of each nozzle 7 intersects the circular edge 13 of the end face 12.

The distance H between the end face 12 of the nozzle 6 and the edge 16 of the front face of each nozzle 7 is about 1.7 mm, for example.

In the embodiment of FIGS. 1 and 2, the nozzles 6 and 7 are constituted by separate fittings.

Naturally, it would not go beyond the ambit of the present invention for the outlet orifices for the substance and for the vector gas to be constituted by the ends of internal passages made in a single piece, as shown in FIG. 3.

In FIG. 3, there can be seen a spray head 20 pierced by lateral passages 21 communicating with the supply of vector gas and a central passage 22 communicating with the supply of liquid substance and opening to the outside via an orifice 24 on the axis X.

The passages 21 open to the outside through distinct orifices 23, on axes Y that intersect the axis X.

In the embodiment of FIG. 3, the vector gas jet emitted by the passages 21 strike each other directly without prior deflection by a deflector-forming element such as the above-mentioned described nozzle 6.

The invention is not limited to dispensing a single liquid substance, and without going beyond the ambit of the present invention it is possible to dispense a mixture of at least two substances, one of the substances being contained in the receptacle containing the vector gas and being entrained therewith, for example.

It is also possible for each of the two substances to be contained in a respective distinct receptacle, both receptacles being different from the receptacle containing the propellant gas.

Under such circumstances, the liquid substances can be fed via two separate paths as shown in FIG. 4.

In FIG. 4, the passage 22 is replaced by two passages 30 and 31 each in communication with a respective one of the two supplies of different substances, e.g. two substances that need to be packaged separately and mixed together extemporaneously.

The axes Z of the orifices 32 and 33 of the passages 30 and 31 are parallel to the spray axis X.

The spray axis X can be horizontal or it can have some other orientation, depending on how the spray device is to be handled and on the location of the zone that is to be treated.

The spray device can be designed to operate head-up or head-down.

The substance can be contained in a removable receptacle, as described below with reference to FIG. 5.

This figure shows a device 40 comprising a receptacle 41 containing the vector gas and a receptacle 42 containing a supply of substance P.

In accordance with the invention, this substance P comprises solid particles and at least one jelling or dispersing agent.

The receptacle 41 is provided with a threaded neck 43.

A spray head 45 is fixed on the hollow control rod 46 of a valve forming part of the receptacle 41.

The receptacle 42 has an annular assembly skirt 47 arranged to screw onto the threaded neck 43.

The receptacle 42 is closed in leakproof manner on top by a cover 48.

The pushbutton 45 presents two vector gas dispensing orifices 48 that are at an angle to each other, having axes Y that intersect each other substantially vertically over a member 50 for feeding the liquid substance and constituted in the example of FIG. 5 by a tube that dips down to the bottom of the receptacle 42.

The tube 50 passes in leaktight manner through the cover 48.

The top end of the tube 50 is provided with an orifice 51 enabling the liquid substance to exit under the effect of the suction created by the vector gas being ejected through the orifices 48 when the user presses on the pushbutton 45.

Having the receptacle 42 fixed removably on the receptacle 41 allows the user to reconstitute the supply of liquid substance once it has been used up, merely by replacing the empty receptacle 42 with a new receptacle full of substance.

Having the receptacle 42 mounted removably on the receptacle 41 also enables the user to select a particular receptacle 42 from a plurality of receptacles containing different substances, e.g. substances of different colors.

Thus, the user can use a single receptacle 41 containing the vector gas in association with a receptacle selected from a plurality of receptacles containing different substances.

It should be observed that the receptacle 42 does not have any air intake orifice other than the passage inside the tube 50.

When the substance is dispensed on actuating the pushbutton 45, suction is established inside the receptacle 42, and when dispensing stops, the return to equilibrium pressure inside the receptacle 42 is accompanied by the substance within the tube 50 moving downwards, thus performing a degree of self-cleaning of the tube 50 and limiting the risk of the tube becoming clogged.

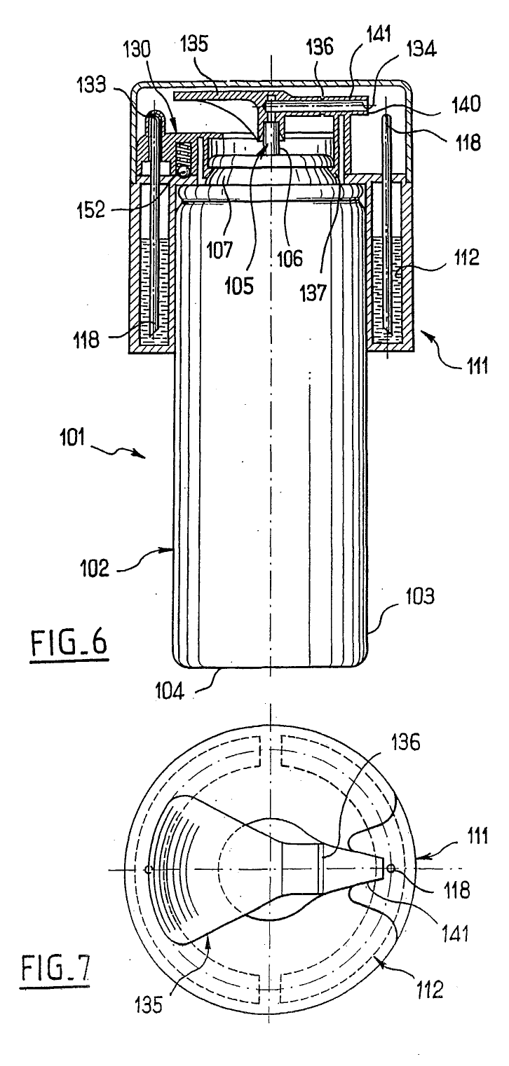

The device 101 shown in FIGS. 6 to 8 is described in detail in French patent application FR-A-2 781 208.

This device 101 comprise a receptacle 102 containing pressurized gas, in particular air. The receptacle 102 is constituted by a can comprising a body 103 having one end closed by a bottom 104. Its end remote from its bottom has a valve 105 mounted thereon, which valve has an emerging rod 106 of the type that is actuated by being pressed down. The valve 105 is carried by a cup 107 fixed on the receptacle 102 by crimping. The valve 105 is itself mounted to the cup 107 by crimping.

An annular tank-carrier member 111 is mounted free to rotate on the receptacle 102 and it defines two tanks 112 each containing a cosmetic for spraying.

A respective duct 118 dips into each tank.

A hoop 130 is snap-fastened onto the receptacle 102.

This hoop 130 is prevented from rotating relative to the receptacle, in particular by being a tight-fit, and it prevents the tank carrier 11 from moving axially.

The hoop 130 has an annular channel 133 suitable for covering the ducts 118 of the tanks 112 so as to isolate them from the outside.

A vector gas dispensing passage 134 is formed inside a projection 141 and opens out laterally through an outlet orifice 140.

The passage 134 is connected to a pushbutton 135 via a film hinge 136 extending perpendicularly to the axis of the receptacle 102.

The outlet orifice 140 is disposed in such a manner as to be capable of being situated as close as possible to the top end of a duct 118. By way of example, this distance can be about 1 mm.

The projection 141 is snap-fastened directly onto the receptacle 102 via a catch 137.

The tank carrier 111 is accurately positioned relative to the outlet orifice 140 for the vector gas by means of an indexing mechanism comprising a ball 152 and a spring, and providing as many indexed positions as there are tanks 112.

Advantageously, the indexed positioning mechanism provides four indexed positions that are spaced apart at 90° intervals: two first positions that are spaced apart by 180° bring the outlet orifice 140 into register with one or other of the ducts 118, and two intermediate or storage positions in which the outlet orifice 140 is not in register with either of the ducts 118, the two intermediate positions being at 90° relative to the first two positions.

Each duct 118 has a bottom end 117 dipping into the bottom of the corresponding tank 112, while its other end emerges from the tank 112.

The passage 134 extends substantially perpendicularly to the axis of each duct 118.

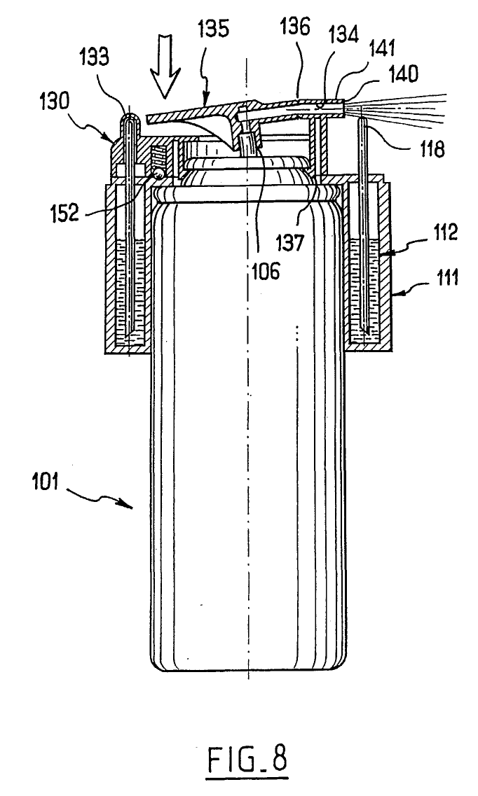

The substance contained in the selected tank 112 whose duct 118 is situated in the vicinity of the orifice 140 is sprayed when the user presses on the pushbutton 135, as shown in FIG. 8.

In the examples described with reference to FIGS. 1 to 8, the substance is sprayed by means of the suction created by the Venturi effect by the vector gas.

Conclusive tests have been performed with the device of FIGS. 1 and 2 using a non-oily foundation makeup having the following composition:

| | |
|---|---|
| BY 29-119 | 40% |
| (cross-linked silicone in aqueous dispersion | |
| from Dow Corning Toray) | |
| Pigments (iron oxide and titanium dioxide) | 7% |
| (solid particles) | |
| Propylene glycol | 7% |
| Glycerin | 4% |
| Carboxymethyl cellulose (jelling agent) | 0.1% |
| Starch powder (solid particles) | 2.5% |
| Hydrophilic modified talc (solid particles) | 2.5% |
| Preservative | q.s. |
| Water | q.s.p. 100% |

To prepare this foundation makeup, the pigments are dispersed in the propylene glycol, the glycerin, and a jell is made with the carboxymethyl cellulose, the water, and the preservative.

The jell is stirred moderately while cold (about 25° C.) for 10 minutes.

The solid particles are dispersed therein, i.e. the BY 29-119 compound, the talc, and the starch while still cold, and the mixture is stirred for 10 minutes.

This provides a foundation that is very fluid, that is stable over time under ordinary conditions of conservation, and in particular for 2 months at 45° C.

Its viscosity is 50 centipoises, measured under the conditions described above.

No clogging of the central nozzle 6 was observed in operation.

Another example of a composition, suitable for body makeup, is as follows:

| | |
|---|---|
| Pigments (solid particles) | 7% |
| Pearlescent agents (solid particles) | 3% |
| Propylene glycol | 2.65% |
| Xanthan (polysaccharide type jelling agent) | 0.5% |
| Pigmented dispersing agents: ARLANTONE 3315 | 0.18% |
| sold by Uniqema (acrylate copolymer and | |
| propylene glycol at 40% in a water and | |
| propylene mixture (50/50), and sodium salt) | |
| BYK-019 | 0.06% |
| Dimethicone copolyl sold by BYK-CERA in | |
| dipropylene glycol monomethyl ether (anti- | |
| toaming agent) | |
| Water | q.s.p. 100% |

Naturally, the invention is not limited to the embodiments described.

In particular, although it is particularly advantageous to use a dispersing agent or a jelling agent in the substance containing solid particles whenever the substance is to be sprayed by means of a self-contained portable device such as any one of those described with reference to FIGS. 1 to 8, the presence of a jelling or dispersing agent in the substance also turns out to be advantageous when the substance is sprayed by being delivered under pressure into a nozzle, with the substance optionally being mixed with a propellant gas.

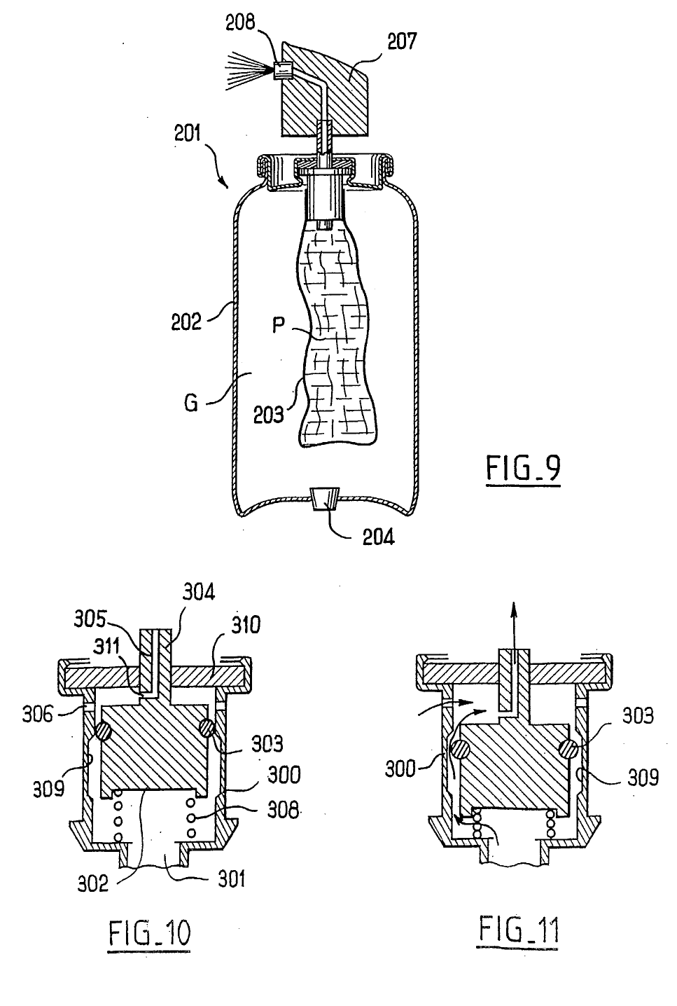

By way of example, FIG. 9 shows a device 201 comprising a receptacle 202 of the aerosol can type, the receptacle containing a bag 203 containing the substance P to be dispensed, and the space outside the bag 203 but inside the receptacle 202 is filled with a gas G, e.g. compressed air.

The bottom of the aerosol can 202 is fitted with a valve 204 enabling it to be refilled with compressed air, and the top of the receptacle 202 includes a valve 205 suitable for dispensing the substance P contained in the bag 203.

The valve 205 has a control rod with a pushbutton 207 carrying a nozzle 208 mounted thereon.

The nozzle 208 can present numerous configurations and it is shown diagrammatically only in order to ensure that the drawing is clear.

When the user presses on the pushbutton 207 and causes the control rod to move downwards, the substance P contained in the bag 203 is delivered under pressure to the nozzle 208, preferably being mixed with gas.

It would not go beyond the ambit of the present invention if the substance were to be sprayed without being mixed with gas.

When the substance is sprayed while mixed with propellant gas, it is possible, for example, to use a valve having at least three positions, as shown diagrammatically in FIGS. 10 and 11, where such a valve is described in great detail in European patent application EP-0 709 305.

This valve comprises a body 300 communicating with the supply of substance at its bottom end via an orifice 301.

A member 302 carrying a sealing ring 303 is movable inside the body 300 against drive from a spring 308.

The member 302 is formed integrally with the control rod 304 of the valve, and is provided with a passage 305 to deliver the substance or the propellant gas.

The passage 305 opens out near the bottom through a lateral orifice 311 which is closed by a gasket 310 when the valve is at rest.

The body 300 is provided with an orifice 306 for admitting propellant gas near the top of the body.

The bag containing the substance is fixed to the body 300 without closing the orifice 306.

The member 302 carries an O-ring 303.

An annular groove 309 is formed in the inside surface of the body 300.

At rest, the O-ring 303 bears against the inside surface of the body 300 above the annular groove 309 so the duct 305 communicates with the inside of the receptacle only through the gas intake orifice 306.

When the control rod 304 is pushed down over a first fraction of its downward stroke, the O-ring 303 continues to bear in leaktight manner against the inside surface of the body 300 while the orifice 311 opens out beneath the gasket 310, thus enabling only propellant gas to pass through the orifice 306, as shown in FIG. 10.

When the control rod 304 is pushed further down, the O-ring 303 reaches the groove 309 and ceases to press against the inside surface of the body 300, thus allowing both substance and propellant gas to pass simultaneously, as shown in FIG. 11.

The position of FIG. 10 can be used to purge the substance contained in the dispenser circuit prior to returning the valve to its rest position.

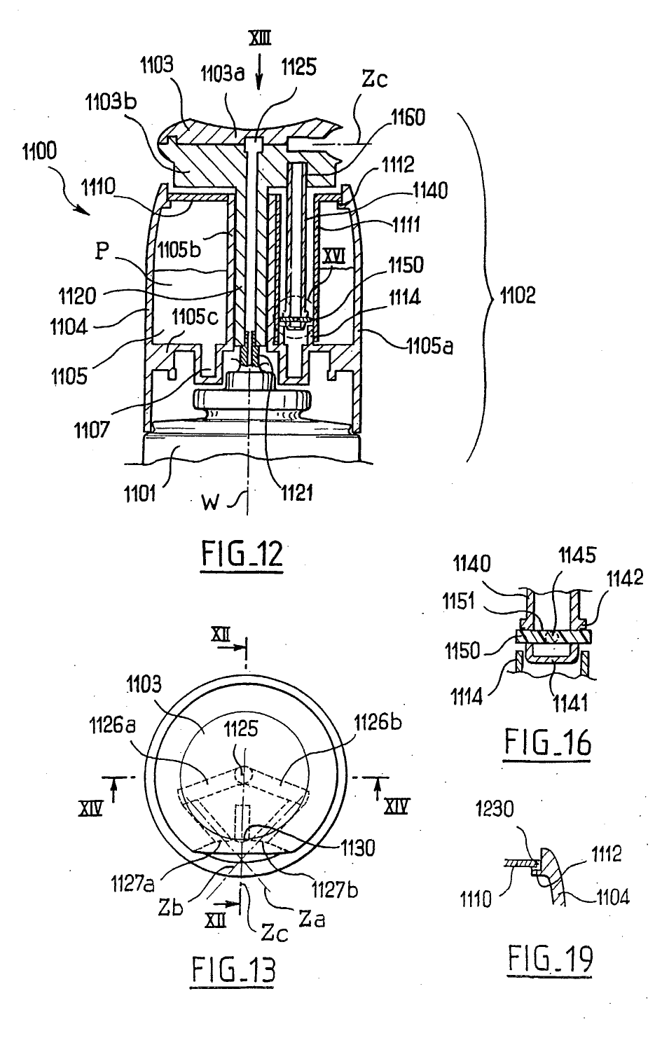

The spray device 1100 shown in FIG. 12 comprises a pressurized receptacle 1101 containing a vector gas in liquefied form, e.g. butane, isopropane, isobutane, or a fluorinated compound, and a dispenser assembly 1102 comprising a pushbutton 1103 and a receptacle 1104 containing a substance P as described above.

In the example described, the receptacle 1104 has a cavity 1105 that is generally annular in shape about an axis W, said cavity 1105 containing the substance P being defined radially on the outside by a first tubular wall 1105a and radially on the inside by a second tubular wall 1105b. The walls 1105a and 1105b are united at the bottom by a bottom end wall 1105c which includes an annular channel 1107 about the axis W, whose function is explained below.

The top of the cavity 1105 is closed by a cover 1110 with a duct 1111 having its axis parallel to the axis W being connected to the bottom face of the cover. The cover 1110 can bear against a shoulder 1112 formed at the top end of the wall 1105a.

The duct 1111 extends over substantially the entire height of the cavity 1105 and its bottom end is assembled to a wall 1114 which projects upwards from the bottom wall 1105c vertically over the channel 1107. The duct 1111 can be assembled to the wall 1114 by engaging one within the other, for example. The above-mentioned wall 1114 can be continuous or discontinuous, and for example it can be constituted by studs.

The duct 1111 and the cover 1110 can be formed integrally, for example.

The pushbutton 1103 is made, for example, by assembling together a top portion 1103a and a bottom portion 1103b. In the example shown, the bottom wall is formed integrally with a hollow rod 1120 for actuating a valve of the pressurized receptacle 1101, the rod being capable of sliding inside the wall 1105b.

This valve can itself have a hollow control rod 1121 engaged in leaktight manner in the bottom end of the rod 1120, and bearing via a shoulder against the bottom end face of the rod 1120.

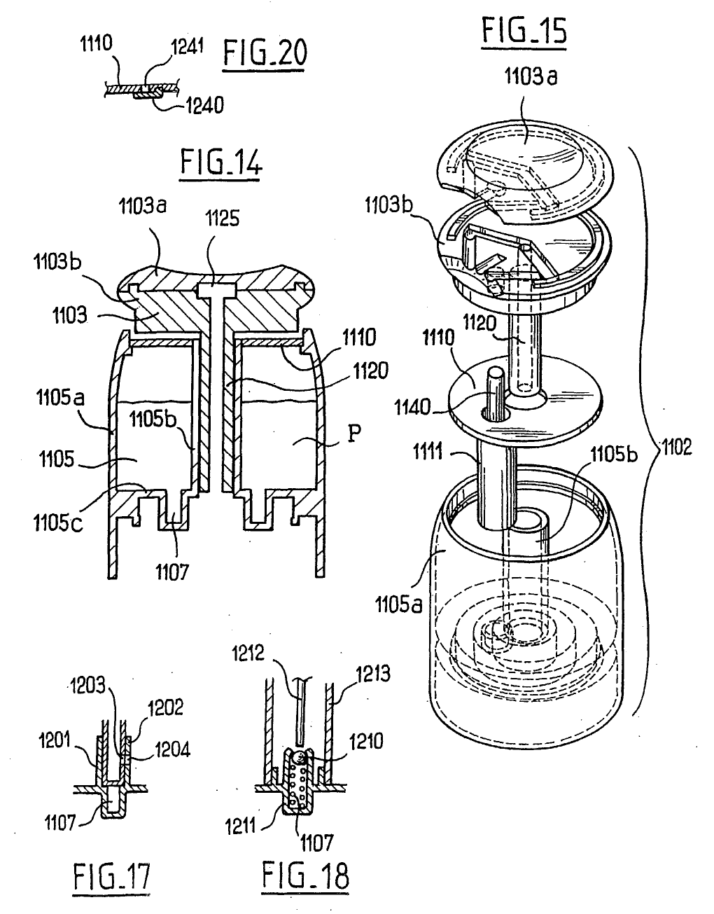

The pushbutton 1103 can be moved along the axis W to act on the control rod 1121, thus causing the vector gas to flow along the duct inside the rod 1120 so as to reach a cavity 1125 which is in communication, as can be seen in FIG. 14, with two internal ducts 1126a and 1126b opening to the outside of the pushbutton via vector gas outlet orifices 1127a and 1127b. The orifices 1127a and 1127b have axes Za and Zb that are substantially perpendicular to each other, for example, each being at an angle of about 45° relative to the spray direction, for example.

The pushbutton 1103 also has a substance feed orifice 1130 having an axis Zc which coincides with the spray direction, for example. The orifice 1130 communicates with the inside of a hollow rod 1140, for example, which rod is closed at its bottom end by a wall 1141, as can be seen in FIG. 16, and on which a shutter-forming annular gasket 1150 is engaged, the gasket 1150 being made of elastomer, for example.

In the example shown, the gasket 1150 bears via its plane top face 1151 against an annular rib 1142 of the rod 1140. The rod has at least one substance admission orifice 1145 whose diameter is less than or equal to the nominal thickness of the gasket 1150 as measured along the axis of the rod 1140. The orifice 1145 is positioned in such a manner that the gasket 1150, when bearing at rest against the rib 1142, covers the orifice 1145 completely and prevents the substance P contained in the cavity 1105 from penetrating via the orifice 1145 into the rod 1140.

By way of example, the rod 1140 is fixed via its top end in a housing 1160 of the pushbutton 1103 and can move together with the rod 1120 when the user presses on the pushbutton 1103. When the rod 1140 is pushed down, the gasket 1150 is compressed between the wall 1114 and the rib 1142 with such compression having the effect of reducing its thickness and releasing the orifice 1145, at least in part, so that the substance contained in the cavity 1105 can flow through the orifice 1145, up the rod 1140, and reach the substance feed orifice 1130.

When the pushbutton 1103 is released, the rod 1140 rises together with the pushbutton so the gasket 1150 can return to a thickness which is sufficient for closing the orifice 1145 because of the gasket's shape memory.

The device 1100 operates as follows. To spray substance P, the user presses on the pushbutton 1103, thereby pushing down the rod 1121 of the valve on the receptacle and causing vector gas to be emitted into the passage inside the rod 1120. The vector gas flows via the duct 1126a and 1126b so as to leave via the orifices 1127a and 1127b, thereby establishing suction in front of the substance feed orifice 1130 by the Venturi effect. pushing down the pushbutton 1103 also has the effect of moving the rod 1140 so as to compress the gasket 1150 as explained above. The orifice 1145 is then released at least in part and substance P contained in the cavity 1105 can rise up the passage inside the rod 1140 and reach the orifice 1130 due to the effect of the above-mentioned suction. The substance is then sprayed along the direction of the axis Zc so long as the user continues to press on the pushbutton 1103. When the pushbutton is released, it can rise back into its rest position because the control rod 1121 is returned into its initial position by resilient means specific to the receptacle 1101, and also because the gasket 1150 has its own elasticity. By returning to its initial shape, the gasket closes the orifice 1145 so that if ever the device 1100 is carried about in a horizontal or an upside-down position, the substance P remains contained inside the cavity 1105 and does not run any risk of leaking out through the substance feed orifice 1130.

By way of example, the receptacle 1104 can be secured removably to the receptacle containing the vector gas, so as to make it possible, where appropriate, to change receptacle 1104 when the supply of substance is used up. This can also enable the receptacle 1101 to be replaced, or enable different substances to be sprayed in succession using a single receptacle 1101.

In the embodiment corresponding to FIGS. 12 to 17, the substance feed is constituted by the end of a duct formed in the pushbutton, however it would not go beyond the ambit of the present invention for the substance feed to comprise a material capable of absorbing the substance by capillarity, e.g. a wick, a felt, or a sintered material.

It is also possible to use a plurality of supplies of different substances mounted in respective chambers of a rotary cylinder, in a manner similar to that described in French patent application FR-A-2 781 208, whose content is incorporated by reference.

Naturally, the invention is not limited to the embodiments described above, and in general, the above-described shutter can be replaced by any suitable shutter means, e.g. shutter means as shown in FIG. 17 comprising two coaxial walls 1201 and 1202 that are movable relative to each other and that constitute a shutter.

By way of example, the inner wall 1202 can be formed by a hollow rod secured to the pushbutton and closed at its bottom end. The substance can rise inside said rod when suction is established by emitting the vector gas.

The outer wall 1201 is stationary and it is suitable for closing an orifice 1203 through the wall 1202 when the pushbutton is at rest. The wall 1202 has an orifice 1204 suitable for coming into register with the orifice 1103 when the pushbutton is pressed down, thereby at least partially releasing the orifice 1203 so as to allow the substance to reach the substance feed.

A check valve, e.g. in the form of a ball, suitable for being opened by moving the pushbutton could also be used, as shown in FIG. 18. Such a check valve can occupy a closed position when the pushbutton is released by the user. In the example shown, the check valve has a shutter-forming ball 1210 urged into a shut position by a spring 1211. An actuator rod 1212 is arranged to be moved downwards when the pushbutton is pushed down. The substance can then flow via a duct 1213 to reach the zone where the suction is established, in order to be sprayed.

In variant embodiments, the annular channel 1107 could be omitted, e.g. if the substance is allowed to reach the space inside the wall 1114, e.g. through openings formed in said wall and in the bottom end of the duct 1111.

It would not go beyond the ambit of the invention for the suction created by emitting the vector gas to be produced through a single orifice.

The control rod 1121 for the receptacle containing the vector gas could also cause the vector gas to be dispensed by being tilted relative to the axis W, providing the pushbutton is designed accordingly.

The receptacle 1104 can be made without air intake or with air intake, in particular if prolonged use is envisaged.

To enable air to be taken in, a microleak 1230 can be made through the cover 1110, for example, as shown in FIG. 19, or between the cover and the wall 1105a of the receptacle 1104, corresponding to the example shown in FIG. 20. Such a microleak is made so as to prevent the substance from passing therethrough, in particular when the device is lying on its side, but to enable air to pass therethrough.

By way of example, a valve 1240 can also be provided that is suitable for closing an air intake orifice 1241, as shown in FIG. 21. The valve 1240 opens in the event of reduced pressure inside the receptacle containing the substance and otherwise closes the orifice 1241.

By way of example, the valve 1240 can be overmolded on the cover 1110, but it could also be made in some other way without going beyond the ambit of the present invention.

1. A self-contained device of unitary structure for spraying a cosmetic substance onto a keratinous medium, in particular the skin, the device comprising a supply of substance to be sprayed and a receptacle containing a gas, together with a valve making it possible, when actuated, to spray the substance, the substance containing solid particles and at least one dispersing or jelling agent.

2. A device according to claim 1, wherein the dispersing or jelling agent is selected so that it does not crystallize at ambient temperature in the medium containing it.

3. A device according to claim 2, wherein the dispersing or jelling agent is selected from agents not included in the following list:
waxes, in particular hydrocarbon waxes or silicone waxes (alkyl or alkoxydimethicone);
fatty acid and fatty alcohol esters, including cerides and sterides or glycerophosphocholine and fatty acid esters, such as phospholipids; and
amino and fatty acids, in particular ceramides.

4. A device according to claim 1, wherein the substance comprises more than 3% by weight of solid particles, preferably 5% to 70% by weight of solid particles, preferably 20% to 70%, more preferably still 30% to 60%, particularly when the substance is a foundation makeup, and wherein the dispersing or jelling agent content lies in the range 0.01% to 10% by weight, and is preferably greater than or equal to 0.1%.

5. A device according to claim 1, wherein the substance includes pigments.

6. A device according to claim 5, wherein the pigments are selected from the following list: iron, titanium, and zinc oxide, organic varnishes, inorganic pearlescent agents, in particular mica, titanium, or titanium-mica, and mixtures thereof.

7. A device according to claim 1, wherein the substance contains particles selected from the following list: organic powders, in particular particles of polyamide, of polyacrylate, of methyl methacrylate, of polyurethane, of polyethylene, of cross-linked polystyrene, and mixtures thereof, powders of mineral origin such as talc, clay, silica, boron nitride and mixtures thereof, powders of vegetable origin such as starch, powders of animal origin such as seashell powder, silicones in the form of beads of methylsesquioxane resin, fluorine-containing powders such as PTFE powders, and mixtures thereof.

8. A device according to claim 1, wherein the particles are of micrometer size (individual grain size).

9. A device according to claim 1, wherein the particles are of nanometer size.

10. A device according to claim 1, wherein the dispersing and/or jelling agent(s) used is/are hydrophilic.

11. A device according to claim 1, wherein the dispersing and/or jelling agent(s) used is/are lipophilic.

12. A device according to claim 10, wherein the substance contains at least one hydrophilic jelling agent selected from: hydrophilic jelling agents of polymeric nature such as jelling agents of polysaccharide type, in particular xanthan gum, gellan gum, guar gum, and derivatives thereof, cellulose and derivatives thereof, jelling agents of vinyl type, carboxyvinyl type, acrylic type, polyurethane, mineral type jelling agents of non-polymeric nature such as clays, in particular Veegum®, optionally modified Bentone®, and hydrophilic silicas, and mixtures thereof.

13. A device according to claim 11, wherein the substance contains a lipophilic jelling agent selected from the following lipophilic jelling agents: polymer type jelling agents or “organo-jelling” type jelling agents, linear or cross-linked silicone polymers and derivatives thereof, polymers derived from silicones, acrylic silicones, polyamide type polycondensates, polysaccharides having hydrophobic chains, and polyurethanes, and mixtures thereof.

14. A device according to claim 1, wherein the substance contains inorganic jelling agents optionally modified by organic groups in order to jell oils.

15. A device according to claim 10, wherein the substance contains at least one dispersing agent and at least one jelling agent.

16. A device according to claim 15, wherein the substance contains at least one hydrophilic dispersing agent selected from the following hydrophilic dispersing agents: dispersing agents having at least one anionic filler, in particular carboxylic dispersing agents, sulfonates, terpolymers of acrylic acid, polyaspartates, derivatives of maleic acid, and mixtures thereof.

17. A device according to claim 1, wherein the substance contains at least one lipophilic dispersing agent selected from the following lipophilic dispersing agents: carboxylates, acrylics, hydrocarbons with a ring, in particular styrene, and hydrocarbons of the succinimide type.

18. A device according to claim 1, wherein the supply of substance is contained in a receptacle that is sufficiently closed to enable the substance to be conserved at ambient temperature in the device when not in use for a length of time in excess of at least 1 month.

19. A device according to claim 1, wherein the viscosity of the substance lies in the range 20 centipoises to 500 centipoises.

20. A device according to claim 1, wherein the substance contains hydrophilic or lipophilic active agents such as, for example, UV blockers (filters), and moisturizers, a care oil, an antioxidant, preserving agents, and anti-foaming agents.

21. A device according to claim 1, wherein the supply of substance is contained in a removable cartridge.

22. A device according to claim 1, wherein the supply of substance is contained in a non-removable receptacle.

23. A device according to claim 1, including a tube dipping into the supply of substance.

24. A device according to claim 1, wherein the supply of substance is contained in a receptacle which is different from the receptacle containing the gas.

25. A device according to claim 1, wherein the substance is contained in a flexible bag placed inside the receptacle containing the gas.

26. A device according to claim 1, wherein the valve has at least three positions, namely: a rest position; a position enabling a mixture of substance and gas to be dispensed; and a position enabling the gas to be dispensed on its own.

27. A device according to claim 1, including at least one substance feed duct and at least one gas feed duct, preferably two gas feed ducts, the ducts being arranged in such a manner that the gas delivered by the feed duct generates a pressure reduction suitable for sucking substance from the substance feed duct.

28. A device according to claim 1, wherein the gas is present in at least a liquid phase inside the receptacle containing the gas.

29. A device according to claim 1, comprising a supply of gas, at least one substance feed suitable for being put into fluid communication with a supply of said substance, the substance being sucked from the supply by suction established in the vicinity of said at least one substance feed by emission of said gas, and at least one shutter suitable for interrupting fluid communication between said at least one substance feed and the supply of substance.

30. A device according to claim 29, wherein the substance feed comprises at least one orifice.

31. A device according to claim 29, the device being arranged in such a manner as to reestablish fluid communication automatically between said at least one substance feed and the supply of substance while gas is being emitted.

32. A device according to claim 1, comprising a pushbutton suitable for acting simultaneously, directly or indirectly, on a gas dispenser valve and on the shutter, whereby fluid communication between said at least one substance feed and the supply of substance is established when the user presses on the pushbutton to cause the gas to be dispensed.

33. A device according to claim 1, comprising a first receptacle fixed on a second receptacle containing the gas.

34. A device according to claim 29, wherein the receptacle containing the substance and the receptacle containing the gas are fixed to each other in such a manner as to prevent them from moving relative to each other in use.

35. A device according to claim 1, wherein the receptacle containing the substance is annular in shape so as to leave a passage through which a control member can extend for controlling a valve fitted to the receptacle containing the gas.

36. A device according to claim 1, wherein the shutter is operationally connected to at least one element actuated by moving the pushbutton of the device.

37. A device according to claim 36, wherein said element comprises a hollow rod having at least one inside passage enabling the substance to reach said at least one substance feed orifice.

38. A device according to claim 1, wherein the shutter comprises at least one gasket suitable for shutting at least one orifice through which the substance can flow to reach said at least one substance feed, and suitable for releasing said orifice at least in part while substance is being dispensed.

39. A device according to claim 38, wherein the shutter is constituted by a gasket mounted on a hollow rod, the rod being closed at its bottom end, the gasket being capable of bearing via its top face against a shoulder of the rod, the rod having at least one substance admission orifice passing therethrough, the orifice being of a diameter that is smaller than or equal to the thickness of the gasket, said gasket being suitable for coming into abutment via its bottom face against a stationary bearing wall when the hollow rod is pushed down, thereby causing the gasket to be compressed so as to release the admission orifice at least in part, thus enabling substance to flow towards said at least one substance feed.

40. A device according to claim 39, wherein the gasket bears via its periphery against the inside surface of a duct in which the hollow rod can move axially, the duct being capable of communicating freely with the outside.

41. A device according to claim 39, wherein the hollow rod is actuated by moving a pushbutton that controls emission of the gas.

42. A device according to claim 39, wherein the space inside the bearing wall against which the gasket can come into abutment via its bottom face communicates with the receptacle containing the substance, via an annular channel formed in a bottom end wall of said receptacle.

43. A device according to claim 1, comprising a pushbutton made by assembling together a bottom portion and a top portion.

44. A device according to claim 43, wherein the bottom portion is made integrally with an actuator rod extending along a passage in the receptacle containing the substance.

45. A device according to claim 44, wherein the control rod of the valve for delivering the gas is engaged in the actuator rod.

46. A device according to claim 29, wherein the gas is emitted via at least one gas outlet orifice.

47. A device according to claim 46, wherein the gas is emitted through at least two gas outlet orifices whose respective positions are selected in such a manner that the gas jets emitted by the orifices meet.
